(19) United States
(12) Patent Application Publication
(10) Pub. No.: US 2005/0059859 A1
(43) Pub. Date: Mar. 17, 2005

(54) Method and system for mounting and securing parts inside an instrument housing

(76) Inventor: Gregory Konstorum, Stamford, CT (US)

Correspondence Address: GANZ LAW, P.C., P O BOX 2200, HILLSBORO, OR, 97123, US

(21) Appl. No.: 10/663,974

(22) Filed: Sep. 15, 2003

(51) Int. Cl. A61B 1/00

(52) U.S. Cl. 600/130

(57) ABSTRACT

A system for mounting a part in an instrument housing includes a mounting surface inside the housing, and at least one resiliently deformable securing element that can removably restrain the part inside the housing so that the part is secured against the mounting surface. The securing element preferably comprises an elastomeric component so that elastic deformation of the component may be used to secure the part and absorb shock. The system is particularly suited for use with medical instruments, particularly, digital endoscopes.

### BACKGROUND OF THE INVENTION

This invention relates to a method and system for removably securing an item of interest inside an instrument housing. More specifically, this invention relates to a method and system for securing and insulating a printed circuit board inside a medical instrument, such as an endoscope.

The ever-growing field of medical instruments for minimally invasive surgery presents several important challenges. For example, instruments are now being made with many components inside a common housing. Precision components, such as printed circuit boards, are among the components typically included in a common housing. Accordingly, there is a need to mount components inside a housing in a manner that protects the components from shock and also secures the components in a desired orientation to assure proper functioning of the instrument. Another need is efficient removal of components for servicing, replacement, upgrading, or cleaning, for example.

Attempts have been made over the years to improve the mounting of components inside instrument housings. In U.S. Pat. No.4,327,738, a part is secured in an endoscope housing using potting material. In U.S. Pat. No. 6,529,232 bellows are welded or soldered in an endoscope. In U.S. Pat. No. 3,539,874 parts are secured by locking tabs. However, none of these designs contemplates the removable securing of the parts, shock protection, or both.

Removability of components is contemplated in some prior art systems by using retention springs and/or fasteners. However, such systems are overly complex and/or do not afford good shock resistance.

Other attempts have been made to improve the shock resistance of the mounting of components in instruments. U.S. Pat. Nos. 4,855,870 and 4,694,555 disclose an assembly for mounting circuit boards in the tubes. A circuit board is mounted to an elongated support spine, which extends longitudinally within a housing tube. A layer of vibration absorbing material is disposed between the circuit board and the spine. In addition, one or more vibration absorbing rubber tubes are placed between the spine and the housing tube. This arrangement of components has shortcomings. For example, there is unnecessary complexity of the components, and the components may be difficult to assemble in an efficient manner. Further, the arrangement may not provide adequate shock absorption due to the way the rubber tubes are arranged in relation to the circuit board.

Another attempt to improve the shock resistance of the mounting of components in a housing is disclosed in U.S. Pat. No. 5,363,276, which discloses a set of grommets used to secure a mounted circuit board in a housing and absorb shock. Each grommet is fastened to a base and receives a notched corner of a board to secure the board to the base in the housing. There are inherent problems with this design. For example, the combination of a grommet and a fastener adds complexity in the number of components used. Further, it may be difficult to align the components during assembly. In addition, this design may add unwanted cost to produce in secondary machining operations, for example, to create appropriate mounting fixtures for the fasteners. There may be inefficiencies during replacement of the components due to lost fasteners, or misalignment.

### SUMMARY OF THE INVENTION

The present invention overcomes the foregoing problems in the prior art by providing a novel system for securing parts inside an instrument housing and providing shock resistance to the mounted parts. The invention particularly relates to removably securing a circuit board in the housing of medical instruments.

According to one possible element of this invention, a part may be placed on a suitable mounting surface, such as opposing shoulders inside an instrument housing. A securing element comprising a resiliently compressible component is disposed adjacent to the part. The securing element may comprise at least one elastomeric component. Possible elastomeric components may be an elastometric hollow tube or a solid cord portion, for example.

To secure the part in the housing and against the opposing shoulders, the securing element may be elastically deformed and inserted in the housing, adjacent to the part. As the securing element reforms, an expansive force results due to diminished dimensions inside the housing and adjacent to the part. This force secures the part on the mounting surface. The same element may also serve as a shock absorber. Multiple elastomeric components can be inserted in this manner, as required by the desired securing force, shape and dimensions inside the housing and of the part, and desired amount of shock absorption, for example.

The foregoing embodiments and features are for illustrative purposes and are not intended to be limiting, persons skilled in the art being capable of appreciating other embodiments from the scope and spirit of the following teachings herein.

### BRIEF DESCRIPTION OF THE DRAWINGS

FIG. 1 shows a front view in cross-section of one embodiment of the present invention.

FIG. 2 shows a sectional front view of an alternate embodiment of the present invention.

FIG. 3a shows a front view of a handle portion of an endoscope of the present invention.

FIG. 3b shows a cross-section of FIG. 3a.

FIG. 3c shows a right side cross-sectional view of FIG. 3a.

FIG. 3d. shows a back view of FIG. 3c.

FIG. 3e shows a detail view of element 11 of FIG. 3d.

FIG. 4. shows a partial cross-sectional view of FIG. 3c.

FIG. 5. shows a section of a partial front view of FIG. 4 along line A-A.

FIG. 6 shows a left side view of a cross section of an endoscope of the present invention.

### DETAILED DESCRIPTION OF THE INVENTION

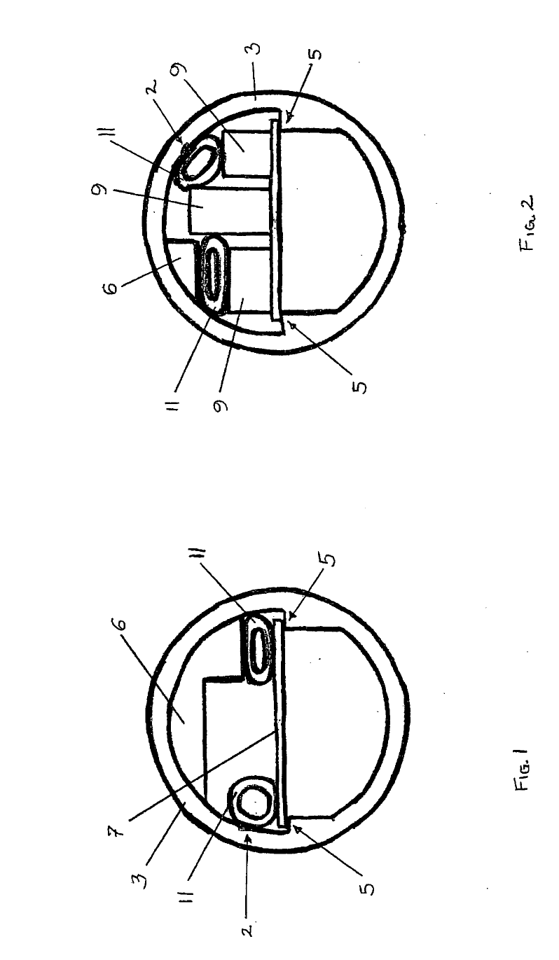

Representative embodiments of the present invention are shown in FIGS. 1-6, wherein similar features share common reference numerals.

The present invention contemplates a system for mounting a part in an instrument housing. A medical endoscope is used to illustrate the invention. A medical endoscope generally includes an elongated tube portion and a proximal a handle member, which may house parts or components. Representative endoscopes are described in U.S. Pat. No. 6,475,140, U.S. patent application Pub. Ser. No. 2001/0031912, and in PCT Pub. No. WO 01/76452, each of which is incorporated herein by reference in its entirety for all purposes.

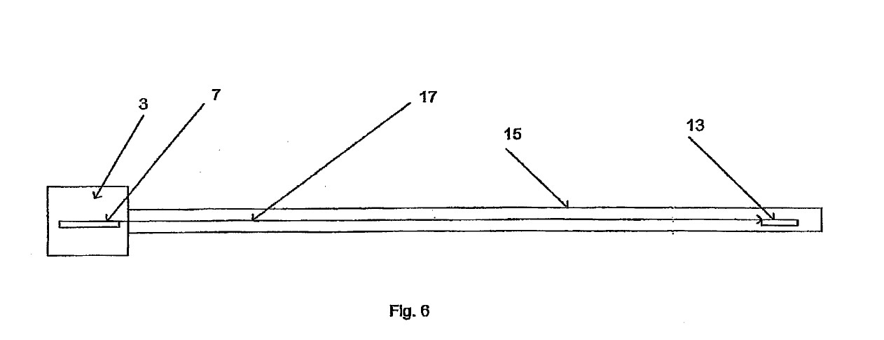

The present invention may be used in both rigid and flexible endoscopes. The present invention is particularly suited for use with digital endoscopes, which may carry on-board electronic components, including image sensors for image acquisition, such as CCD and CMOS image sensors, and LED or other solid-state lighting systems for illuminating a surgical site or other target site. For example, FIG. 6 shows an endoscope having a housing 3 and an elongated tube portion 15. The tube portion 15 may be generally hollow and adapted to receive components, such as, for example, a light guide and working channel. A communication conduit 17 may be used to link a CMOS image sensor, or other electronic component, to a part 7 in the housing 3. Hereinafter “part” and “component” are used interchangeably and may refer to a single part or an assembly of a plurality of parts.

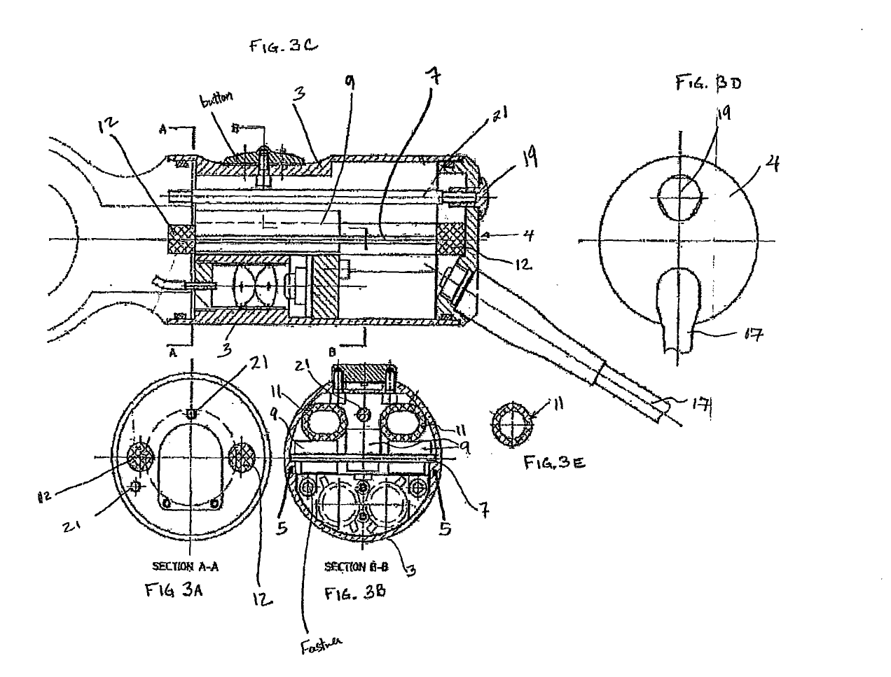

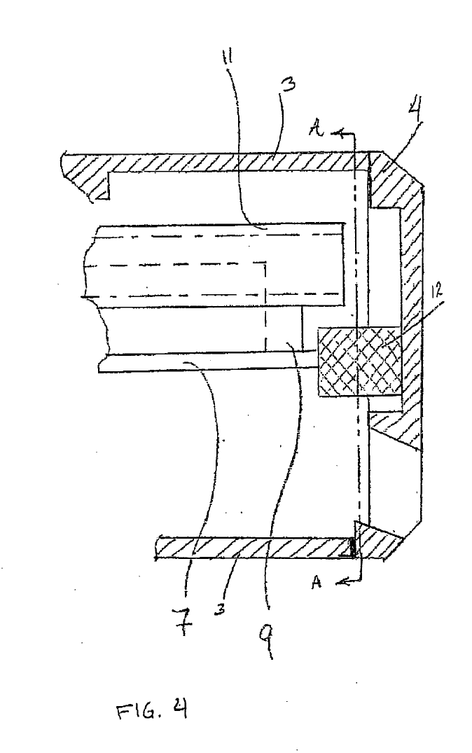

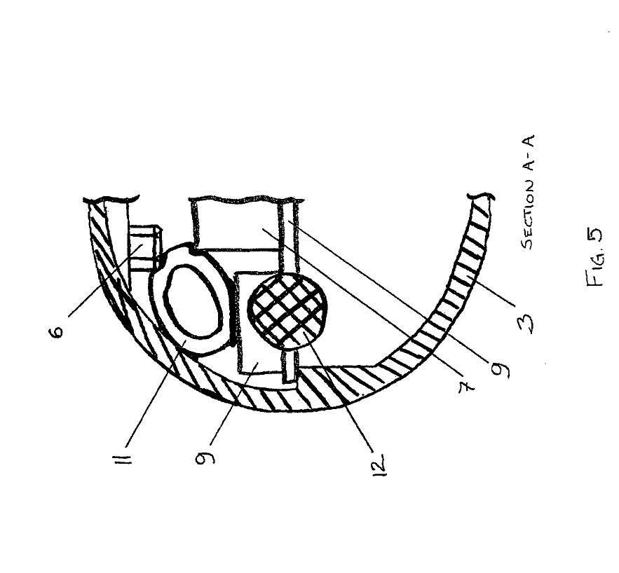

Referring to FIGS. 3a and 3b, housing 3 has an internal mounting surface or surfaces 5. A mounting surface 5 may be disposed in the housing in a manner that facilitates removable mounting of a part. One possible example of a mounting surface or surfaces 5, as shown in the figures, is a pair of shoulders disposed longitudinally on opposing internal sidewalls of the housing 3. The shoulders may extend radially from the walls sufficiently to provide supporting surfaces for a part. The shoulders allow, for example, a planar part 7 having a width greater than the space between the shoulders to be slidably fitted into the housing over the shoulders. The mounting surface 5 is not limited to the foregoing, and it may be any surface that provides desired support for a generally planar part 7. For instance, instead of opposing shoulders, the supporting surface may be disposed along any surface along a chord of the housing. The surface may lie in two or three dimensions so long as it can support a predetermined part 7.

The invention contemplates the use of a resiliently deformable securing element that can removably restrain the part 7 against the mounting surface, such as opposing shoulders 5. The deformable securing element can removably restrain the part inside the housing by, for example, using the elastic deformation of an elastomeric component so that force for reformation of the component causes the part to be secured against the mounting surface. More particularly, retention of the part 7 in the body member 3 may be accomplished by squeezing, or otherwise causing a resiliently deformable securing element to deform to a size less than that provided in the housing, thus allowing space to fit the part on top of the mounting surface 5 and part 7. A securing force is created by an interference fit of the elastomeric component against the part and corresponding interior features 6 of the housing 3, for example. As shown in FIGS. 1, 2 and 5, the securing element may contact the part 7 directly and at the same time contact an inside surface of the housing 3 and/or features 6 in the housing. Features 6 may be, for example, structural elements inside the housing 3 or other components mounted to interior portions of the housing 3. In a preferred embodiment illustrated by the right securing element 11 in FIG. 1, the securing element or elements are essentially “wedged” or compressed to fit inside the housing and generally disposed adjacent to one or more features in an interference fit. In an alternative embodiment illustrated by the left securing element 11 in FIG. 1, instead of an interference fit, the securing element or elements may be affixed to the interior of the housing using, for example, an adhesive or fastener 2.

Referring now to FIGS. 3-4, in one embodiment, a combination of longitudinally disposed securing elements 11 are used in combination with end portions of securing elements 12. However, alternative embodiments contemplate any combination of elements 12 and 11. An elongated securing element 11 is adapted for use in combination with the shoulders 5, as shown in the figures, while other elements 12 may be placed between the housing 3 and/or end cap 4. For example, FIG. 3c illustrates one contemplated arrangement of securing elements 12 in relation to the board 7, the housing 3, and end cap 4. For instance, a shock absorbing element, such as a hollow tube or solid cord element 12, may be placed between a part 7 and an end cap 4 of the housing 3. In this manner, any clearance space between a proximal end of the part 7 and the housing 3, including the end cap 4, may be filled by the element 12. Similarly, at a distal end of the part 7, a second element 12 may be positioned adjacent to the distal end, so that any clearance space between the part 7 and the housing 3 at the distal end may be filled with the securing element 12. Any combination of quantity, size, and hardness of elements 12 may be used to provide the desired securing and shock-resisting properties for a given application. Thus, the part 7 may be mounted in the housing in a secure and shock-resisting manner by the use of end located elements 12.

The deformable securing elements 11 and 12 also provide shock resistance using known, resiliently deformable energy absorbing materials or structures. Possible materials for elements 11 and 12 may be a piece of an elastomeric material, such as a tube or cord made from silicone, rubber, foam or other elastic material or spring structure, for example. In a preferred embodiment, material for elements 11 and 12 would have a durometer hardness rating of about 40 A to about 70 A, as commonly measured on a Shore test durometer. If a hollow member, for example, element 11, has a given hardness and diameter, a solid member, for example, element 12, of the same hardness would be less flexible and more rigid due to the relative amount of material in a given cross-sectional diameter. Any combination of either hardness of the element, size of the element, or element configuration, including pluralities of elements, may be used to provide the desired securing and shock absorbing characteristics. A preferred embodiment includes two elongated and hollow securing elements 11 placed between the housing and running parallel to shoulder features 5 and against the board 7. In one embodiment, at least two solid cord members, securing elements 12, are placed at opposing ends of the part, such as board 7. One element 12 is disposed between a distal end of the housing 3 and the board 7, while an oppositely placed second element 12 is placed at a proximal end and disposed between the board 7 and the end cap 4. This provides added securing force and protection from shock forces traveling axially along the instrument.

A securing element may be wholly or partly adjacent or connected to the housing 3, as shown in FIGS. 1 and 2, or it may be wholly or partly adjacent an intermediate structure 15, as shown in FIGS. 3-5. Also, while the figures show element 1I1 as a hollow tube member, and element 12 as a solid diameter cord member, either element 11 or 12 may be hollow or solid, in any combination.

To remove the part 7 from the body member 3, a deforming pressure may be exerted on the securing element 11 so that sufficient clearance exists between the part 7 and the element 11, thus facilitating extraction from the mounting surface 5 inside the body member 3.

In a preferred embodiment an end cap 4 may be attached to a housing 3 by using one or more fasteners 19, which may be threaded to a corresponding standoff 21. As shown in FIG. 3c, a securing element 12 may be positioned at opposite ends of the board 7 so that fastening the end cap 4 to the housing would compress the elements 12, thus securing the board 7 in place.

Persons skilled in the art will recognize that many modifications and variations are possible in the details, materials, and arrangements of the parts and actions which have been described and illustrated in order to explain the nature of this invention and that such modifications and variations do not depart from the spirit and scope of the teachings and claims contained herein.

1. A system for removably mounting a part in a housing, the system comprising:
a mounting surface inside the housing;
at least one resiliently deformable securing element for securing the part inside the housing by applying a force causing the part to be secured against the mounting surface.

2. The system of claim 1 wherein the mounting surface comprises:
opposing shoulders inside the housing for supporting a substantially planar part.

3. The system of claim 1 wherein the securing element is in direct contact with the part and a second structure in the housing.

4. The system of claim 2 wherein the part comprises a printed circuit board.

5. The system of claim 2 wherein the securing element comprises an elongate elastomeric material.

6. The system of claim 2 wherein the securing element comprises a tubular element.

7. An assembly for mounting a circuit board in a medical instrument housing, comprising:
a housing;
a circuit board;
at least one deformable securing element that can removably secure the circuit board inside the housing so that the circuit board rests on mounting surfaces disposed in the housing and comprising a pair of shoulders; and
the deformable securing element being adjacent to the printed circuit board.

8. The assembly housing of claim 7 wherein the deformable securing element comprises an elongate elastomeric material.

9. An assembly for an endoscope, the assembly comprising:
a mounting surface on an interior portion of a proximal end portion of the endoscope;
a substantially planar part disposed on the mounting surface; and
at least one resiliently deformable securing element that applies a force to removably secure the part on the mounting surface and provide protection against shock.

10. The assembly of claim 9 wherein the mounting surface is a pair of opposing shoulders disposed oppositely along surfaces of the interior portion of the endoscope.

11. The instrument housing of claim 9 wherein the substantially planar part is a printed circuit board.

12. The instrument housing of claim 9 wherein the endoscope includes a solid-state lighting system.

13. The instrument housing of claim 9 wherein the endoscope includes solid-state image sensor.

14. A method for removably securing a part in a medical instrument housing comprising the steps of:
providing a hollow instrument housing having an internal mounting surface;
inserting the part in the housing so that it is disposed on the mounting surface;
providing a deformable securing element;
compressing the securing element; and
arranging the securing element to secure the part against the mounting surface, part being removably secured by the force from the deformation of the securing element.

15. The method of claim 14 wherein the part is inserted in an endoscope.

16. The method of claim 14 wherein the part is a circuit board.

17. The method of claim 15 wherein the endoscope includes a solid-state image sensor.

18. The method of claim 15 wherein the endoscope includes a solid-state lighting system.

19. The method of claim 15 wherein the mounting surface comprises a pair of shoulders oppositely disposed along interior surfaces of the housing.

20. The method of claim 17 wherein the endoscope includes a solid-state lighting system.
